(19) United States
(12) Patent Application Publication
(10) Pub. No.: US 2022/0208356 A1
(43) Pub. Date: Jun. 30, 2022

(54) Radiological Based Methods and Systems for Detection of Maladies

(71) Applicant: CareMentor AI LLC, Moscow (RU)

(72) Inventors: Pavel Roytberg, Moscow (RU); Dmitriy Blinov, Moscow (RU); Evgeniy Zhukov, Moscow (RU)

(21) Appl. No.: 17/563,903

(22) Filed: Dec. 28, 2021

Related U.S. Application Data
(60) Provisional application No. 63/131,758, filed on Dec. 29, 2020.
(60) Provisional application No. 63/220,768, filed on Jul. 12, 2021.
(60) Provisional application No. 63/290,448, filed on Dec. 16, 2021.

(51) Int. Cl. G16H 30/40; G16H 50/20; A61B 6/00

(52) U.S. Cl. CPC G16H 30/40; A61B 6/5217; A61B 6/50; G16H 50/20

(57) ABSTRACT

The present disclosure generally relates to a system and method for determining medical maladies in radiological imaging. In exemplary embodiments, neural network models may be established to read radiological images for characteristics associated with for example, pulmonary diseases. Embodiments may build predictions from an ensemble of model outputs that predict various pulmonary characteristics from features identified in the images. The system may be used to process a selected patient's X-ray lung image to make predictions of whether pulmonary disease is present in the image.

### CROSS-REFERENCE TO RELATED APPLICATIONS

This application claims the benefit of U.S. Provisional Patent Application No. 63/131,758, filed Dec. 29, 2020, titled “Method and Apparatus For Screening Lung Pathology”, U.S. Provisional Patent Application No. 63/220,768, titled “Radiological Based Methods and Systems For Detection of Maladies,” filed Jul. 12, 2021, and U.S. Provisional Patent Application No. 63/290,448, titled “Radiological Based Methods and Systems For Detection of Maladies,” filed Dec. 16, 2021.

This application incorporates the entire contents of the above-referenced applications herein by reference.

### TECHNICAL FIELD

The present disclosure is directed to radiological based methods and systems for detection of maladies.

### BACKGROUND OF THE DISCLOSURE

In conventional practice, the analysis of radiological images (for example, from X-rays or CT scans), requires manual review of the images. A physician discerns from images which often have hazy outlines of matter, elements of the image that may be of concern. However, it is possible to mistake noisy elements in the image for elements indicative of a characteristic of concern. The images may be so grainy or the contrast so difficult to interpret, that sometimes another image must be taken. In addition, the volume of images that need to be studied may sometimes overwhelm a physician's available time.

The exemplary disclosed system and method of the present disclosure is directed to overcoming one or more of the shortcomings set forth above and/or other deficiencies in existing technology.

### SUMMARY

According to an embodiment of the subject technology, a computer program product for determining radiological signs of pulmonary disease is disclosed. The computer program product comprises one or more computer readable storage media, and program instructions collectively stored on the one or more computer readable storage media. The program instructions comprise: receiving by a computer processor, images of one or more lungs from different patients; building a neural network model for identifying pulmonary related pathologies from the images of one or more lungs from an ensemble of model outputs; generating from the neural network model, a multi-class classification of pulmonary related pathologies; receiving from a selected patient, a selected image of the patient's lungs; providing the selected image of the patient's lungs to the neural network model; identifying features in the selected image; and determining by the neural network model, whether any identified features in the selected image are predicted to be a pulmonary disease.

According to another embodiment, a method for determining radiological signs of pulmonary disease is disclosed. The method includes: receiving by a computer processor, images of one or more lungs from different patients; building a neural network model for identifying pulmonary related pathologies from the images of one or more lungs from an ensemble of model outputs; generating from the neural network model, a multi-class classification of pulmonary related pathologies; receiving from a selected patient, a selected image of the patient's lungs; providing the selected image of the patient's lungs to the neural network model; identifying features in the selected image; and determining by the neural network model, whether any identified features in the selected image are predicted to be a pulmonary disease.

According to yet another embodiment, a computer server for determining radiological signs of pulmonary disease is disclosed. The computer server includes a network connection; one or more computer readable storage media; a processor coupled to the network connection and coupled to the one or more computer readable storage media; and a computer program product comprising program instructions collectively stored on the one or more computer readable storage media. The program instructions include: receiving by the computer processor, images of one or more lungs from different patients; building a neural network model for identifying pulmonary related pathologies from the images of one or more lungs from an ensemble of model outputs; generating from the neural network model, a multi-class classification of pulmonary related pathologies; receiving from a selected patient, a selected image of the patient's lungs; providing the selected image of the patient's lungs to the neural network model; identifying features in the selected image; and determining by the neural network model, whether any identified features in the selected image are predicted to be a pulmonary disease.

The techniques described herein may be implemented in a number of ways. Example implementations are provided below with reference to the following figures.

### BRIEF DESCRIPTION OF THE DRAWINGS

Accompanying this written specification is a collection of drawings of exemplary embodiments of the present disclosure. One of ordinary skill in the art would appreciate that these are merely exemplary embodiments, and additional and alternative embodiments may exist and still within the spirit of the disclosure as described herein.

FIGS. 1A-1B are a flowchart of data flow for pulmonary disease detection, in accordance with at least some exemplary embodiments of the present disclosure;

FIGS. 2A-2B are a flowchart of a process using neural modeling for pulmonary disease detection, in accordance with at least some exemplary embodiments of the present disclosure

FIG. 3 is a schematic illustration of an exemplary computing device, in accordance with at least some exemplary embodiments of the present disclosure;

FIG. 4 is a schematic illustration of an exemplary network, in accordance with at least some exemplary embodiments of the present disclosure; and

FIG. 5 is a schematic illustration of an exemplary network, in accordance with at least some exemplary embodiments of the present disclosure.

### DETAILED DESCRIPTION AND INDUSTRIAL APPLICABILITY

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. Like or similar components are labeled with identical element numbers for ease of understanding.

In general, and referring to the Figures, embodiments of the subject technology provide neural network modeling approaches to detect pulmonary disease from images. In exemplary embodiments, neural network models may be established to read radiological images for characteristics associated with for example, pulmonary diseases. Embodiments may build predictions from an ensemble of model outputs that predict various pulmonary characteristics from features identified in the images. The system may be used to process a selected patient's X-ray lung image to make predictions of whether pulmonary disease is present in the image.

As will be appreciated the amount of data to process in the features extracted in radiological images from the corpora of images used to build the neural network models is beyond the reasonable capability of an individual or a team of individuals. The number of features related to pulmonary maladies potentially present in any one image may be extensive and identifying each feature it is not practically possible for a given accuracy and/or timeframe by human capacity. Accordingly, the time required to identify all possible features for one selected image, much less a corpus of images, may exceed the time needed to act on a patient's condition when an element contributing to disease is present. Aspects of the subject technology provide a technical benefit of reducing the time requirements and computational loads of an appropriately configured computing platform for determining when features indicating a probability of pulmonary disease is present in an image.

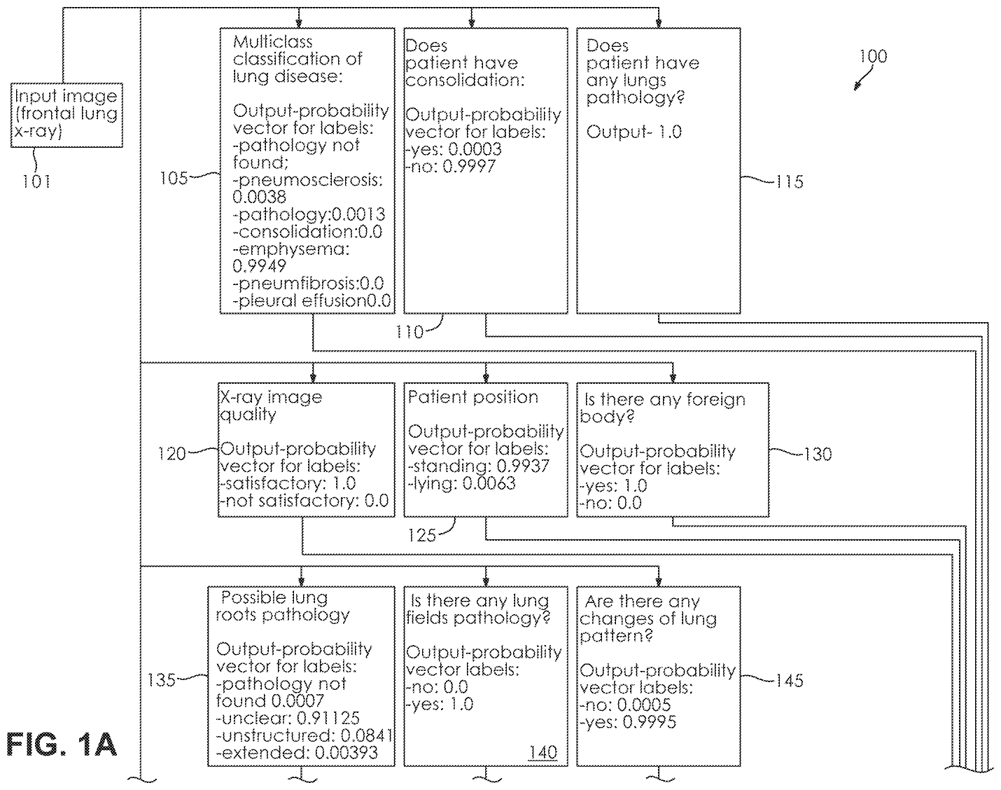

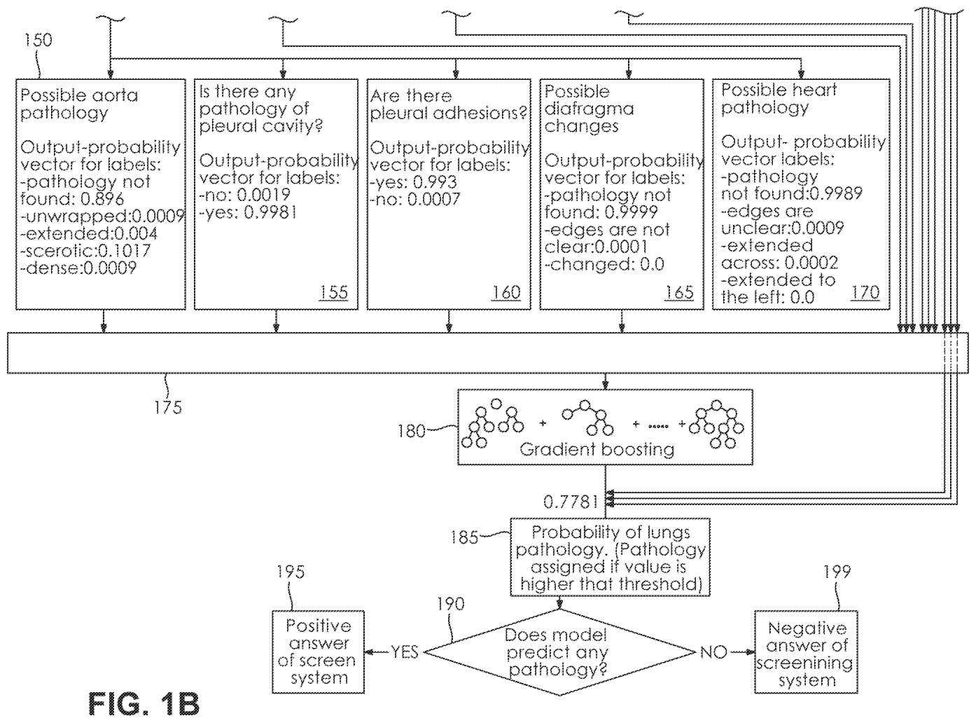

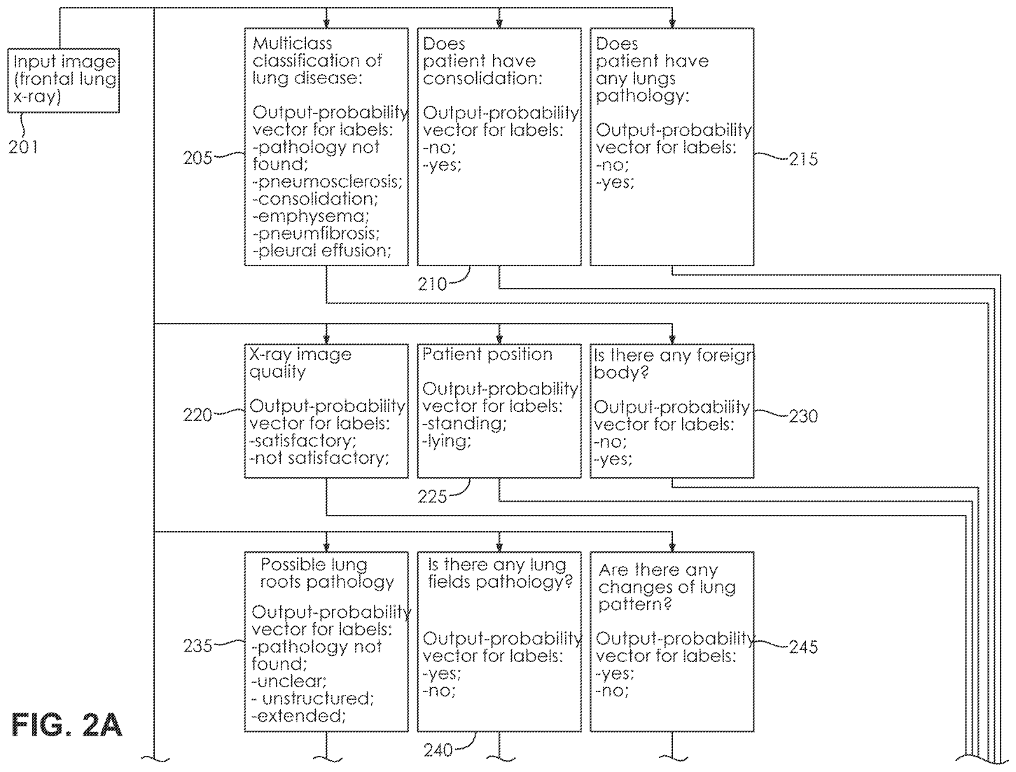

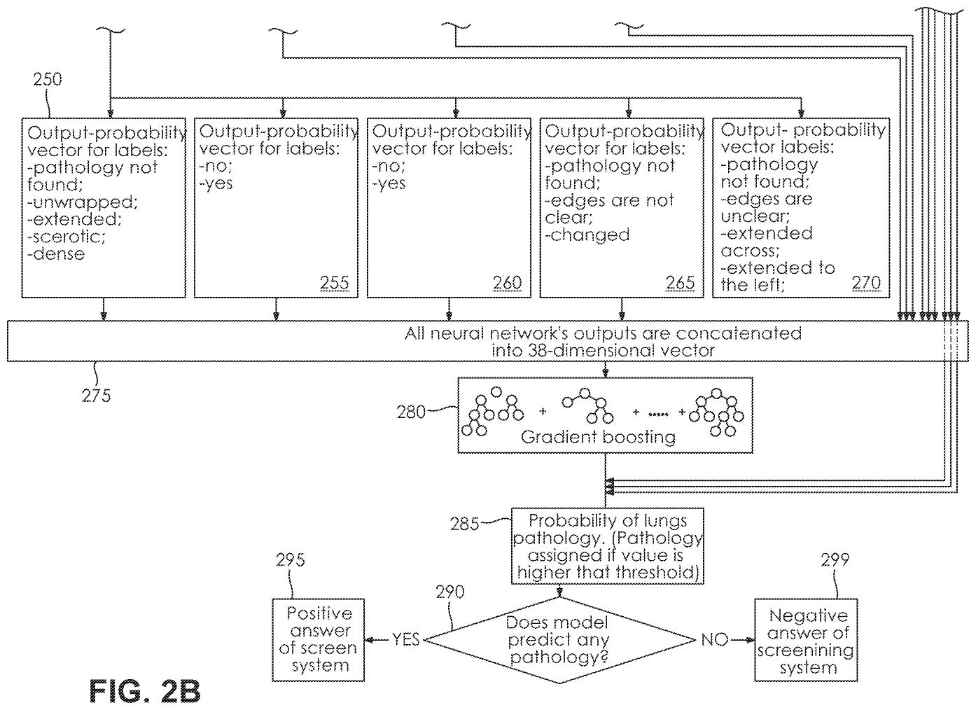

Referring now to FIGS. 1A-1B and 2A-2B, an approach to screening the chest for lung pathology is proposed. FIGS. 1A-1B shows a process 100 for detecting pulmonary disease according to embodiments. FIGS. 2A-2B shows a process 200 using neural modeling for detecting pulmonary disease as an embodiment.

Referring now to FIGS. 1A-1B, the process 100 for detecting pulmonary disease is shown according to an illustrative embodiment. In one embodiment, an ensemble of neural network models and a gradient boosting model built on the ensemble outputs may be combined to identify one or more pulmonary diseases present in X-ray images of lungs. The ensemble of models may comprise, for example, fourteen neural networks. Each neural network may be independently trained on its own unique subset of data, with its own features of data preparation and features of the learning algorithm. In FIGS. 1A-1B, the output of respective blocks 105-170 may represent the ensemble of models.

In the process 100 shown, some blocks have numbers present for different characteristics or findings. The numbers are provided as possible percentages associated with the characteristic or criterium in the block and are illustrative only as examples that a neural model may use in processing certain features for determining outputs/labels from the input data. Generally, the process 100 includes receiving 101 input images of lung X-rays. In some embodiments, the images are taken from the frontal perspective of lungs. The input images are processed 105 by the neural network modeling 175 through a multiclass classification of lung diseases. In block 110 the modeling 175 may determine whether a patient's images has consolidation. In block 115, the modeling 175 may determine whether the patient images show any lung pathology. In some embodiments, the X-ray image quality may be used as a constraint to determine 120 a reliability of the labels found for an image. Some embodiments may use a patient's positioning in an image as a constraint in determining 125 the probability vectors for labels found in an image. Some embodiments may check 130 whether there are any foreign bodies found in an image. In an exemplary embodiment, the modeling 175 may determine 135 whether any possible lung roots pathology is present in one or more images. In some embodiments, the modeling 175 may determine 140 whether lung fields pathology was present in one or more images. In some embodiments, the modeling 175 may determine 145 whether there are any changes in lung patterns present between different images. In some embodiments, the modeling 175 may determine 150 whether one or more images show any features associated with aorta pathology. In some embodiments, the modeling 175 may determine 155 whether one or more images show any features associated with pleural cavity pathology. In some embodiments, the modeling 175 may determine 160 whether one or more images show any features associated with pleural adhesions. In some embodiments, the modeling 175 may determine 165 whether images show changes to the diaphragm between images. In some embodiments, the modeling 175 may determine 170 whether one or more images show any features associated with heart pathology.

Some embodiments may include a block incorporating 180 gradient boosting of the data processed by the neural network model 175. The process 100 may include a probability output generated 185 of whether a pathology is found present in one or more images. Embodiments may determine 190 whether the probability of a pathology being present based on a threshold value being met or exceeded. If the neural model predicts 190 a pathology is present, a positive answer of is generated 195 in the system as an output. If the probability does not predict a pathology, a negative answer may be generated 199.

FIGS. 2A-2B show a process 200, which is similar to the process 100 except that instead of percentages and values being used in the respective individual blocks, labels are substituted. The individual blocks in FIGS. 2A-2B share the same numbering as FIGS. 1A-1B except that FIGS. 2A-2B use the 200 series.

The processes described above were found to produce the following illustrative results:

In some embodiments, a set of three neural network models may be assembled and designed to classify a specific disease (for example, out of five possible diagnoses), or the absence thereof. An exemplary embodiment may include the following neural models:

A classification model for the classification of pneumosclerosis, consolidation, emphysema, pulmonary fibrosis, pleural effusion, or the absence of these pathologies.

A separate model trained to identify signs of consolidation, or the absence thereof.

A separate model trained on an expanded set of various chest organ pathologies to obtain a binary conclusion about the presence of a pathology or the absence thereof.

Three descriptive models, which may include:
a model to assess the analyzed image quality (satisfactory or not);
a model to identify the patient's position (for example, whether the study was performed in a standing or lying position); and
a model to determine foreign bodies in the area under study.

Three models for diagnosing the pathology of individual lung structures, which may include for example:
a model for binary classification of the presence or absence of pulmonary field pathologies;
a model for the classification of possible pathologies of the lung hilum (dilated, not clearly visualized, poorly structured, or no pathology); and
a model to identify changes in the lung pattern;

Five models for diagnosing pathologies of other adjacent areas and organs of the chest, which may include:
a model for binary classification of presence or absence of pleural cavity pathologies;
a separate model to identify pleural adhesions;
a model for the classification of possible pathologies of the aorta (for example identifying a dilated, a sclerosed, an indurated, a reversed aortic arch, or no pathologies identified);
a model to identify changes in the diaphragm (for example, where the diaphragm boundaries are not clearly visualized, other changes, or is unremarkable); and
a model to identify pathologies in the heart area (for example, the borders of the heart are not clearly visualized, the heart can be dilated in diameter, to the left, or no significant changes found).

Each of the ensemble models allows classifying the analyzed study according to one or another attribute. However, to make a final conclusion on whether the patient has signs of lung pathologies, it is necessary to take into account the response of all models simultaneously. For this purpose, a gradient boosting model was trained on trees, the output value for which is a 38-dimensional numerical vector obtained by combining outputs from all ensemble models. The model output is the probability that the patient has signs of pathology. The final decision of the model is determined by comparing the model output with a certain threshold value selected in such a way as to minimize the number of false-negative responses, while not significantly increasing the number of false-positive responses.

Now, having three trained neural network models designed directly for the classification of some specific diseases, as well as a gradient boosting model, the final decision about the absence of signs of any pulmonary pathology in the patient should be made only if the output of each of the 4 models corresponds to the “no pathology detected” class. Otherwise, a decision on the presence of signs of pulmonary pathology in the patient should be made.

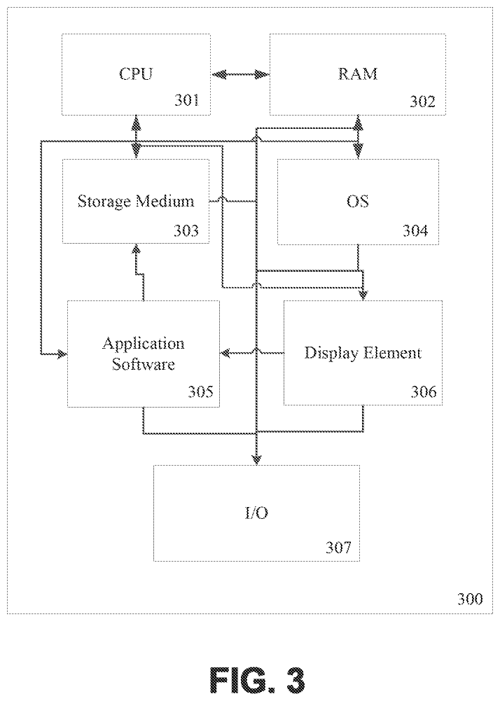

Referring now to FIG. 3, an illustrative representation of a computing device 300 appropriate for use with embodiments of the system of the present disclosure is shown. The computing device 300 may be representative of a machine that controls and performs one or more of the neural network modeling and processes for identification of maladies in radiology and X-ray imaging as described above. The computing device 300 can generally be comprised of a Central Processing Unit (CPU, 301), optional further processing units including a graphics processing unit (GPU), a Random Access Memory (RAM, 302), a mother board 103, or alternatively/additionally a storage medium (e.g., hard disk drive, solid state drive, flash memory, cloud storage), an operating system (OS, 304), one or more application software 305, a display element 306, and one or more input/output devices/means 307, including one or more communication interfaces (e.g., RS232, Ethernet, Wifi, Bluetooth, USB). Useful examples include, but are not limited to, personal computers, smart phones, laptops, mobile computing devices, tablet PCs, and servers. Multiple computing devices can be operably linked to form a computer network in a manner as to distribute and share one or more resources, such as clustered computing devices and server banks/farms.

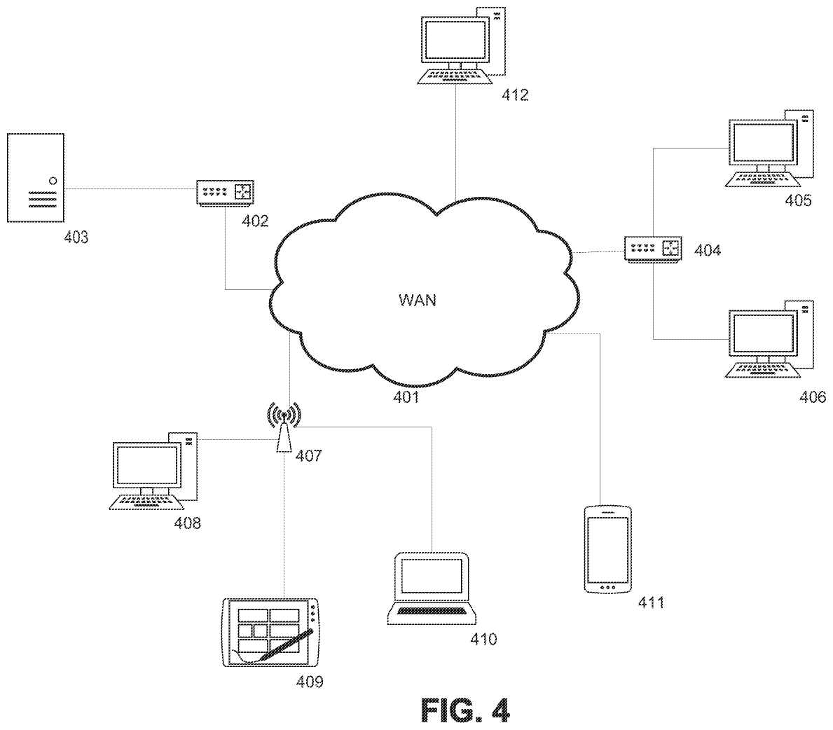

Various examples of such general-purpose multi-unit computer networks suitable for embodiments of the disclosure, their typical configuration and many standardized communication links are well known to one skilled in the art, as explained in more detail and illustrated by FIG. 4, which is discussed herein-below.

According to an exemplary embodiment of the present disclosure, data may be transferred to the system, stored by the system and/or transferred by the system to users of the system across local area networks (LANs) (e.g., office networks, home networks) or wide area networks (WANs) (e.g., the Internet). In accordance with the previous embodiment, the system may be comprised of numerous servers communicatively connected across one or more LANs and/or WANs. One of ordinary skill in the art would appreciate that there are numerous manners in which the system could be configured and embodiments of the present disclosure are contemplated for use with any configuration.

In general, the system and methods provided herein may be employed by a user of a computing device whether connected to a network or not. Similarly, some steps of the methods provided herein may be performed by components and modules of the system whether connected or not. While such components/modules are offline, and the data they generated will then be transmitted to the relevant other parts of the system once the offline component/module comes again online with the rest of the network (or a relevant part thereof). According to an embodiment of the present disclosure, some of the applications of the present disclosure may not be accessible when not connected to a network, however a user or a module/component of the system itself may be able to compose data offline from the remainder of the system that will be consumed by the system or its other components when the user/offline system component or module is later connected to the system network.

Referring to FIG. 4, a schematic overview of a system in accordance with an embodiment of the present disclosure is shown. The system is comprised of one or more application servers 403 for electronically storing information used by the system. Applications in the server 403 may retrieve and manipulate information in storage devices and exchange information through a WAN 401 (e.g., the Internet). Applications in server 403 may also be used to manipulate information stored remotely and process and analyze data stored remotely across a WAN 401 (e.g., the Internet). The applications in server 403 may be configured to generate a neural network model of the types described above or generate determinations as discussed above.

According to an exemplary embodiment, as shown in FIG. 4, exchange of information through the WAN 401 or other network may occur through one or more high speed connections. In some cases, high speed connections may be over-the-air (OTA), passed through networked systems, directly connected to one or more WANs 401 or directed through one or more routers 402. Router(s) 402 are completely optional and other embodiments in accordance with the present disclosure may or may not utilize one or more routers 402. One of ordinary skill in the art would appreciate that there are numerous ways server 403 may connect to WAN 401 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 403 via WAN 401 or other network in numerous ways. For instance, a component or module may connect to the system i) through a computing device 412 directly connected to the WAN 401, ii) through a computing device 405, 406 connected to the WAN 401 through a routing device 404, iii) through a computing device 408, 409, 410 connected to a wireless access point 407 or iv) through a computing device 411 via a wireless connection (e.g., CDMA, GMS, 3G, 4G, 5G) to the WAN 401. One of ordinary skill in the art will appreciate that there are numerous ways that a component or module may connect to server 403 via WAN 401 or other network, and embodiments of the present disclosure are contemplated for use with any method for connecting to server 403 via WAN 401 or other network. Furthermore, server 403 could be comprised of a personal computing device, such as a smartphone, acting as a host for other computing devices to connect to.

The communications means of the system may be any means for communicating data, including image and video, over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

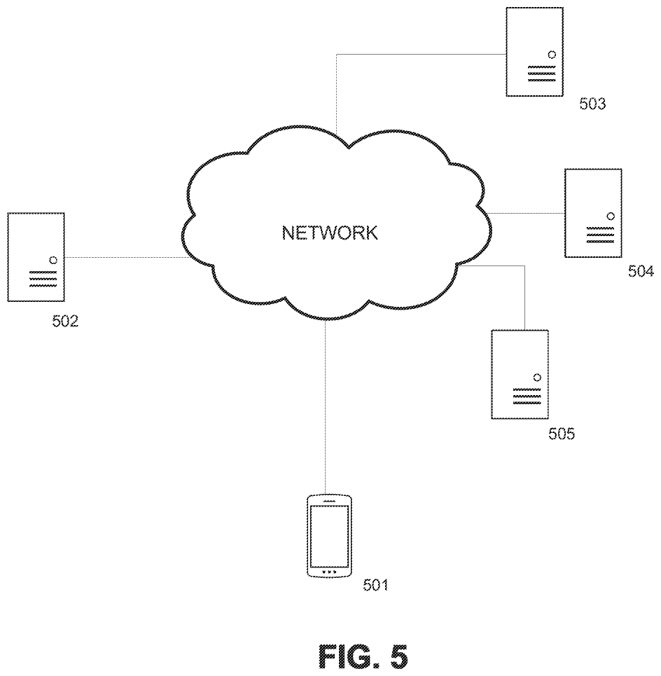

Turning now to FIG. 5, a continued schematic overview of a cloud-based system in accordance with an embodiment of the present invention is shown. In FIG. 5, the cloud-based system is shown as it may interact with users and other third party networks or APIs (e.g., APIs associated with the exemplary disclosed E-Ink displays). For instance, a user of a mobile device 501 may be able to connect to application server 502. Application server 502 may be able to enhance or otherwise provide additional services to the user by requesting and receiving information from one or more of an external content provider API/website or other third party system 503, a constituent data service 504, one or more additional data services 505 or any combination thereof. Additionally, application server 502 may be able to enhance or otherwise provide additional services to an external content provider API/website or other third party system 503, a constituent data service 504, one or more additional data services 505 by providing information to those entities that is stored on a database that is connected to the application server 502. One of ordinary skill in the art would appreciate how accessing one or more third-party systems could augment the ability of the system described herein, and embodiments of the present invention are contemplated for use with any third-party system.

Traditionally, a computer program includes a finite sequence of computational instructions or program instructions. It will be appreciated that a programmable apparatus or computing device can receive such a computer program and, by processing the computational instructions thereof, produce a technical effect.

A programmable apparatus or computing device includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computing device can include any and all suitable combinations of at least one general purpose computer, special-purpose computer, programmable data processing apparatus, processor, processor architecture, and so on. It will be understood that a computing device can include a computer-readable storage medium and that this medium may be internal or external, removable and replaceable, or fixed. It will also be understood that a computing device can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the disclosure as claimed herein could include an optical computer, quantum computer, analog computer, machine learning, artificial intelligence computations, or the like.

Regardless of the type of computer program or computing device involved, a computer program can be loaded onto a computing device to produce a particular machine that can perform any and all of the depicted functions. This particular machine (or networked configuration thereof) provides a technique for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Illustrative examples of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A data store may be comprised of one or more of a database, file storage system, relational data storage system or any other data system or structure configured to store data. The data store may be a relational database, working in conjunction with a relational database management system (RDBMS) for receiving, processing and storing data. A data store may comprise one or more databases for storing information related to the processing of moving information and estimate information as well one or more databases configured for storage and retrieval of moving information and estimate information.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software components or modules, or as components or modules that employ external routines, code, services, and so forth, or any combination of these. All such implementations are within the scope of the present disclosure. In view of the foregoing, it will be appreciated that elements of the block diagrams and flowchart illustrations support combinations of means for performing the specified functions, combinations of steps for performing the specified functions, program instruction technique for performing the specified functions, and so on.

It will be appreciated that computer program instructions may include computer executable code. A variety of languages for expressing computer program instructions are possible, including without limitation C, C++, Java, JavaScript, assembly language, Lisp, HTML, Perl, and so on. Such languages may include assembly languages, hardware description languages, database programming languages, functional programming languages, imperative programming languages, and so on. In some embodiments, computer program instructions can be stored, compiled, or interpreted to run on a computing device, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computing device enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computing device can process these threads based on priority or any other order based on instructions provided in the program code.

Unless explicitly stated or otherwise clear from the context, the verbs “process” and “execute” are used interchangeably to indicate execute, process, interpret, compile, assemble, link, load, any and all combinations of the foregoing, or the like. Therefore, embodiments that process computer program instructions, computer-executable code, or the like can suitably act upon the instructions or code in any and all of the ways just described.

The functions and operations presented herein are not inherently related to any particular computing device or other apparatus. Various general-purpose systems may also be used with programs in accordance with the teachings herein, or it may prove convenient to construct more specialized apparatus to perform the required method steps. The required structure for a variety of these systems will be apparent to those of ordinary skill in the art, along with equivalent variations. In addition, embodiments of the disclosure are not described with reference to any particular programming language. It is appreciated that a variety of programming languages may be used to implement the present teachings as described herein, and any references to specific languages are provided for disclosure of enablement and best mode of embodiments of the disclosure. Embodiments of the disclosure are well suited to a wide variety of computer network systems over numerous topologies. Within this field, the configuration and management of large networks include storage devices and computing devices that are communicatively coupled to dissimilar computing and storage devices over a network, such as the Internet, also referred to as “web” or “world wide web”.

In at least some exemplary embodiments, the exemplary disclosed system may utilize sophisticated machine learning and/or artificial intelligence techniques to prepare and submit datasets and variables to cloud computing clusters and/or other analytical tools (e.g., predictive analytical tools) which may analyze such data using artificial intelligence neural networks. The exemplary disclosed system may for example include cloud computing clusters performing predictive analysis and determinations identifying medical conditions. For example, the exemplary neural network may include a plurality of input nodes that may be interconnected and/or networked with a plurality of additional and/or other processing nodes to determine a predicted result. Exemplary artificial intelligence processes may include filtering and processing datasets, processing to simplify datasets by statistically eliminating irrelevant, invariant or superfluous variables or creating new variables which are an amalgamation of a set of underlying variables, and/or processing for splitting datasets into train, test and validate datasets using at least a stratified sampling technique. The exemplary disclosed system may utilize prediction algorithms and approach that may include regression models, tree-based approaches, logistic regression, Bayesian methods, deep-learning and neural networks both as a stand-alone and on an ensemble basis, and final prediction may be based on the model/structure which delivers the highest degree of accuracy and stability as judged by implementation against the test and validate datasets.

Throughout this disclosure and elsewhere, block diagrams and flowchart illustrations depict methods, apparatuses (e.g., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on—any and all of which may be generally referred to herein as a “component”, “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present disclosure are contemplated for use with any language.

While multiple embodiments are disclosed, still other embodiments of the present disclosure will become apparent to those skilled in the art from this detailed description. There may be aspects of this disclosure that may be practiced without the implementation of some features as they are described. It should be understood that some details have not been described in detail in order to not unnecessarily obscure the focus of the disclosure. The disclosure is capable of myriad modifications in various obvious aspects, all without departing from the spirit and scope of the present disclosure. Accordingly, the drawings and descriptions are to be regarded as illustrative rather than restrictive in nature.

What is claimed is:

1. A computer program product for determining radiological signs of pulmonary disease, the computer program product comprising:
one or more computer readable storage media, and program instructions collectively stored on the one or more computer readable storage media, the program instructions comprising:
receiving by a computer processor, images of one or more lungs from different patients;
building a neural network model for identifying pulmonary related pathologies from the images of one or more lungs from an ensemble of model outputs;
generating from the neural network model, a multi-class classification of pulmonary related pathologies;
receiving from a selected patient, a selected image of the patient's lungs;
providing the selected image of the patient's lungs to the neural network model;
identifying features in the selected image; and
determining by the neural network model, whether any identified features in the selected image are predicted to be a pulmonary disease.

2. The computer program product of claim 1, wherein the program instructions further comprise determining by the neural network model whether the selected image shows lung consolidation.

3. The computer program product of claim 1, wherein the program instructions further comprise performing gradient boosting of the ensemble of model outputs.

4. The computer program product of claim 1, wherein the program instructions further comprise receiving an image quality of the selected image and factoring in the image quality in the prediction of the pulmonary disease.

5. The computer program product of claim 1, wherein the program instructions further comprise receiving a position of the patient in the selected image and factoring in the position of the patient in the prediction of the pulmonary disease.

6. The computer program product of claim 1, wherein the program instructions further comprise determining whether a foreign body is present in the selected image and factoring in the presence of the foreign body in the prediction of the pulmonary disease.

7. The computer program product of claim 1, wherein the selected image of the patient's lungs is an X-ray image.

8. A method for determining radiological signs of pulmonary disease, comprising:
receiving by a computer processor, images of one or more lungs from different patients;
building a neural network model for identifying pulmonary related pathologies from the images of one or more lungs from an ensemble of model outputs;
generating from the neural network model, a multi-class classification of pulmonary related pathologies;
receiving from a selected patient, a selected image of the patient's lungs;
providing the selected image of the patient's lungs to the neural network model;
identifying features in the selected image; and
determining by the neural network model, whether any identified features in the selected image are predicted to be a pulmonary disease.

9. The method of claim 8, further comprising determining by the neural network model whether the selected image shows lung consolidation.

10. The method of claim 8, further comprising performing gradient boosting of the ensemble of model outputs.

11. The method of claim 8, further comprising receiving an image quality of the selected image and factoring in the image quality in the prediction of the pulmonary disease.

12. The method of claim 8, further comprising receiving a position of the patient in the selected image and factoring in the position of the patient in the prediction of the pulmonary disease.

13. The method of claim 8, further comprising determining whether a foreign body is present in the selected image and factoring in the presence of the foreign body in the prediction of the pulmonary disease.

14. The method of claim 8, wherein the selected image of the patient's lungs is an X-ray image.

15. A computer server for determining radiological signs of pulmonary disease, comprising:
a network connection;
one or more computer readable storage media;
a processor coupled to the network connection and coupled to the one or more computer readable storage media; and
a computer program product comprising program instructions collectively stored on the one or more computer readable storage media, the program instructions comprising:
receiving by the computer processor, images of one or more lungs from different patients;
building a neural network model for identifying pulmonary related pathologies from the images of one or more lungs from an ensemble of model outputs;
generating from the neural network model, a multi-class classification of pulmonary related pathologies;
receiving from a selected patient, a selected image of the patient's lungs;
providing the selected image of the patient's lungs to the neural network model;
identifying features in the selected image; and
determining by the neural network model, whether any identified features in the selected image are predicted to be a pulmonary disease.

16. The computer server of claim 15, wherein the program instructions further comprise determining by the neural network model whether the selected image shows lung consolidation.

17. The computer server of claim 15, wherein the program instructions further comprise performing gradient boosting of the ensemble of model outputs.

18. The computer server of claim 15, wherein the program instructions further comprise receiving an image quality of the selected image and factoring in the image quality in the prediction of the pulmonary disease.

19. The computer server of claim 15, wherein the program instructions further comprise receiving a position of the patient in the selected image and factoring in the position of the patient in the prediction of the pulmonary disease.

20. The computer server of claim 15, wherein the program instructions further comprise determining whether a foreign body is present in the selected image and factoring in the presence of the foreign body in the prediction of the pulmonary disease.
